(19) United States
(12) Patent Application Publication
(10) Pub. No.: US 2014/0377829 A1
(43) Pub. Date: Dec. 25, 2014

(54) METHOD AND SYSTEM FOR PRODUCING BIOGAS

(71) Applicants: Jarmo Jarvinen, Lahti (FI); Niklas Tornkvist, Kauniainen (FI)

(72) Inventors: Jarmo Jarvinen, Lahti (FI); Niklas Tornkvist, Kauniainen (FI)

(73) Assignee: MAASEUDUN VOIMA OY, LAHTI (FI)

(21) Appl. No.: 14/376,169

(22) PCT Filed: Feb. 1, 2013
(86) PCT No.: PCT/FI2013/050112

§ 371 Date: Aug. 1, 2014

(30) Foreign Application Priority Data
Feb. 1, 2012 (FI) 20125110

(51) Int. Cl. C12P 5/02; C12M 1/34; C12M 1/00

(52) U.S. Cl. CPC C12P 5/023; C12M 29/04; C12M 41/26
USPC 435/167; 435/297.1; 435/286.1

(57) ABSTRACT

A system (100, 200, 300, 400) for producing biogas from waste stream (101) including organic material includes a liquid waste container (102) and a solid waste container (103). In addition, it includes a separator (104) for at least partially separating liquid fraction into the liquid waste container (102) and solid fraction into the solid waste container (103) from the waste stream (101) so that the solid waste container includes more solid content than the liquid waste container. Furthermore, the system includes a communication member (106, 111) between the liquid waste container (102) and a mixer (105, 202) for introducing liquid from the liquid waste container to the waste stream portion containing solid fraction in order to extract more liquid from the solid fraction.

### TECHNICAL FIELD OF THE INVENTION

The invention relates to a method and system for producing biogas, such as e.g. methane, hydrogen, carbon dioxide, and/or combinations thereof, from a waste stream, like from organic feedstock.

### BACKGROUND OF THE INVENTION

Numbers of solutions are known from prior art for producing biogas from organic feedstock, such as from livestock faeces and urine. Different kinds of treatments for producing biogas from organic feedstock are used, such as keeping and heating the organic feedstock in fermentation tanks as well as utilizing anaerobic digestion. Typical prior art process takes about 30 to 60 days to produce biogas. However, these kinds of methods are not very powerful taking quite long time, and in addition very large fermentation tanks are needed to store the huge amount of livestock faeces and urine produced in 30-60 days for example in a cattle farm with 100 animals.

In addition it is known [JP2000263018A] to separate the organic waste into liquid waste and solid waste portions and sent liquid waste portion to a first methane fermentation tank and solid waste portion to a second methane fermentation tank to produce biogas by two fermentation tanks. Again it is known that the methane fermentation of the liquid waste can be carried out even in 2 to 4 days. There are still however some disadvantages relating to the known prior art, such as the amount of the produced biogas is quite low compared to the initial amount of the organic waste before the separation, for example.

### SUMMARY OF THE INVENTION

An object of the invention is to alleviate and eliminate the problems relating to the known prior art. Especially the object of the invention is to provide a system for producing very effectively and fast biogas from biomass containing waste streams and organic feedstock, such as from livestock faeces and urine.

The object of the invention can be achieved by the features of independent claims.

The invention relates to a system for producing biogas according to claim 1. In addition the invention relates to a module for producing biogas from a waste stream comprising biomass according to claim 18 as well as to a method according to claim 19.

According to the invention biogas is produced from organic feedstock or waste stream comprising any biomass, such as waste streams from cattle, pig or poultry farms, industrial or municipal waste water streams, waste foodstuffs or organic waste water, such as sewage, livestock manure, faeces and urine, for example. Biogas produced is for example methane, hydrogen, carbon dioxide, or combinations thereof.

According to the invention liquid fraction of the waste stream is at least partially separated into a liquid waste container (e.g. a 1^{st} methane fermentation tank) and solid fraction into a solid waste container (e.g. 2^{nd} methane fermentation tank) so that after separation the solid waste container comprises more solid content than the liquid waste container. According to an embodiment liquid from the liquid waste container is introduced (recycled) to the waste stream portion in order to extract more liquid from the solid fraction of the waste stream portion. The introduction point may be before the separation point, especially if the waste stream is dry, like is the case for example with poultry waste, for example. As well the introduction point may be after the separation point or in connection with the solid waste container, which is the case with the waste stream with a great excess of liquid before separation, like is the case with cattle waste, for example. It is to be noted that also inoculum material can be added to introduce bacteria and other substances as a culture medium and thereby triggering and strengthen the fermentation process already from the beginning. As an example said inoculum material can be achieved for example from the liquid waste container.

The present invention offers clear advantages over the known prior art. By introducing some liquid and/or inoculum material into the solid waste stream or into the solid waste container, more liquid can be extracted from the overall content of the waste stream into the liquid (fermentation) container, starting of the fermentation process can triggered and advanced and thereby more powerful and faster fermentation and biogas production is achieved. This also minimizes the container volume requirements. In addition the invention offers an arrangement for producing and collecting biogas, which can be easily extended depending on the amount of the raw waste stream feedstock. Modularity of the systems enables a scalable system with low costs and with minimum interference for the regular farm operations.

The novel features which are considered as characteristic of the invention are set forth in particular in the appended claims. The invention itself, however, both as to its construction and its method of operation, together with additional objects and advantages thereof, will be best understood from the following description of specific embodiments when read in connection with the accompanying drawings.

The exemplary embodiments of the invention presented in this patent application are not to be interpreted to pose limitations to the applicability of the appended claims. The verb “to comprise” is used in this patent application as an open limitation that does not exclude the existence of also unrecited features. The features recited in depending claims are mutually freely combinable unless otherwise explicitly stated.

### BRIEF DESCRIPTION OF THE DRAWINGS

Next the invention will be described in greater detail with reference to exemplary embodiments in accordance with the accompanying drawings, in which:

FIGS. 1-4 illustrates an exemplary method and system for producing biogas according to an advantageous embodiment of the invention, and

FIG. 5 illustrates an exemplary diagram of methane fermentation rates according to an embodiment of the invention versus a prior art method.

### DETAILED DESCRIPTION

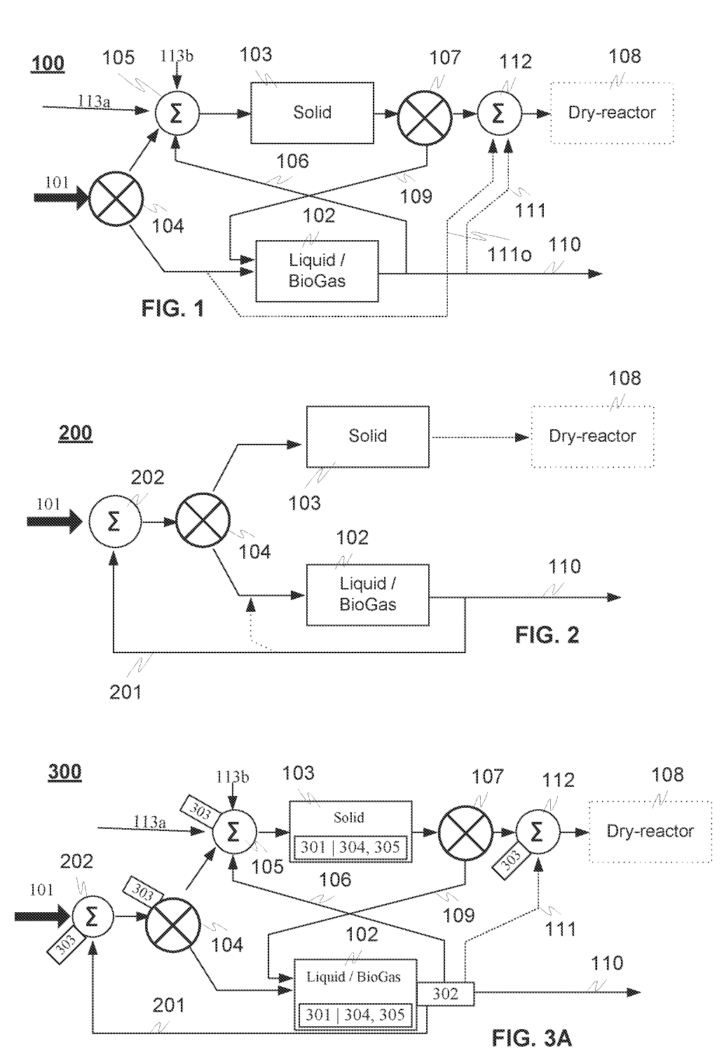

FIG. 1 illustrates an exemplary method and system 100 for producing biogas from waste stream 101, where the original waste stream feedstock comprises a great excess of liquid, like is the case with cattle waste for example. The system advantageously comprises at least one liquid waste container 102 (liquid reactor) for liquid fractions, and at least one solid waste container 103 (solid reactor) for solid fractions. The system comprises also a first separator 104 for at least partially separating liquid fraction from the waste stream 101 into the liquid waste container 102 (e.g. 1^{st} methane fermentation tank) and solid fraction from the waste stream 101 into the solid waste container 103 (e.g. 2^{nd} methane fermentation tank). After separation said solid waste container 103 advantageously comprises more solid content than the liquid waste container 103.

According to an embodiment the system 100 also comprises a first mixer 105 and a communication means 106, such as a pipe, between the liquid waste container 102 and the mixer 105. The communication means 106 is provided for introducing (recycling) a portion of liquid from the liquid waste container 102 to the waste stream portion comprising solid fraction in order to extract more liquid from said solid fraction. The first mixer 105 is advantageously arranged before the solid waste container 103, whereupon the communicated liquid from the liquid waste container 102 is introduced via the mixer 105 into the solid waste container 103, where the extraction advantageously happens. The extracted liquid may then be separated from the waste stream portion after the solid waste container 103 by the second separator 107, whereafter the excess of the waste stream portion (especially the solid content of it) is transferred into a dry type reactor 108.

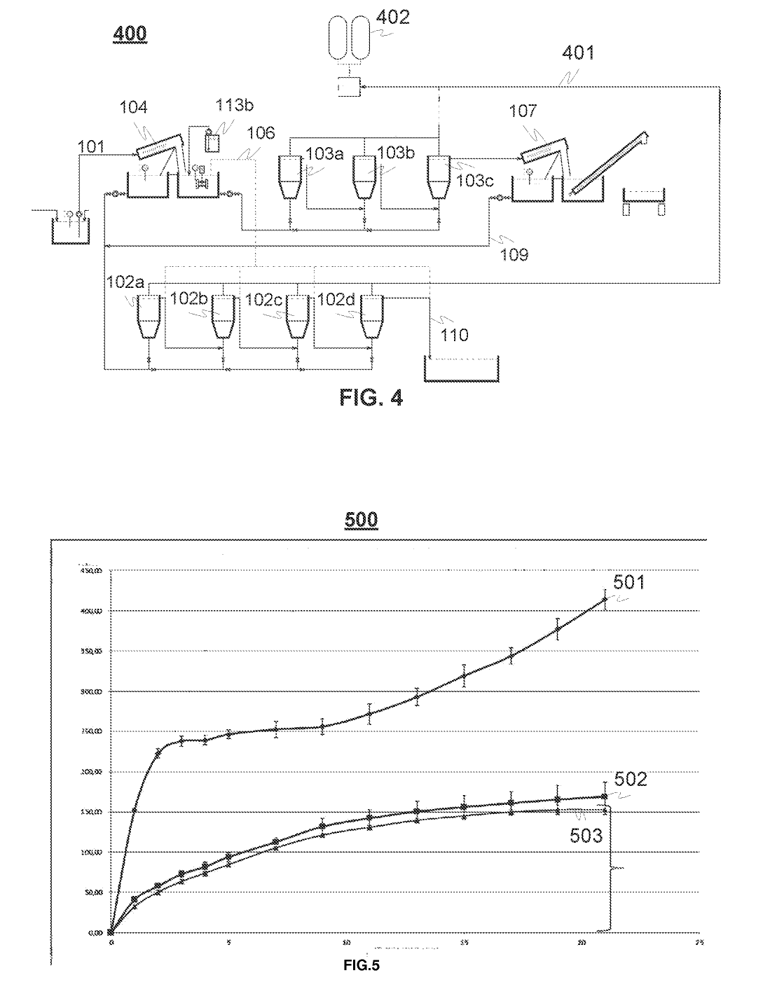

According to an embodiment of FIG. 1 the extracted liquid is introduced into the liquid waste container 102 via a communication means 109, but it to be noted that the extracted liquid may as well be introduced into another liquid waste container being in another state of fermentation and having liquid with different concentration (as is the case with FIG. 5).

In addition the system comprises communication means 110 (like a pipe) for transferring excess of the liquid waste stream from the liquid waste container 102 either to a subsequent liquid waste container (as is the case with FIG. 5) or out from the system, such as into a waste water treatment station. The system may optionally comprise also communication means 111 for introducing a liquid fraction from the liquid waste container 102 or communication means 111 directly after the separator 104 into the solid waste stream via a second mixer 112 before transferring it into the dry type reactor for adjusting pH of the stream or using the liquid as buffering pH, for example.

Furthermore it is to be noted that the system may comprise additional inlets 113a, 113b arranged for example in the connection with the firs mixing means in order to introduce for example additional organic material 113a into the waste stream, or introducing acid (such as solution of sulfuric acid, NaOH, Ca(OH)₂), enzyme or other biocatalyst 113b for example to break down any cellulose materials within solid fraction. The additional organic waste material may be e.g. grass, reed, creed canary grass, or the like. Even though the additional inlets 113a, 113b are described into the connection with the first mixer 105, they may also be arranged into another point of the system as well.

Advantages of the embodiment described in FIG. 1 are that by introducing some liquid into the solid waste stream or into the solid waste container, more liquid can be extracted from the overall content of the waste stream into the liquid (fermentation) container 102 and thereby more powerful fermentation and biogas production is achieved.

The mixers may be implemented e.g. by different kinds of pipe joints and valves for example, and they may be provided either before the separator (like is the case with poultry waste, for example) or after the separator and before the solid waste container (like is the case with cattle waste, for example). Of course the mixers may also comprise mixing means, such as propeller or blender in order to mix said components of streams better to each other.

It is to be noted that the biogas production is mainly managed in the liquid waste container 102, even though some biogas production occurs also in the solid state container 103. However, the production rate is much more effective in the liquid waste container 102, as can be seen in connection with FIG. 5.

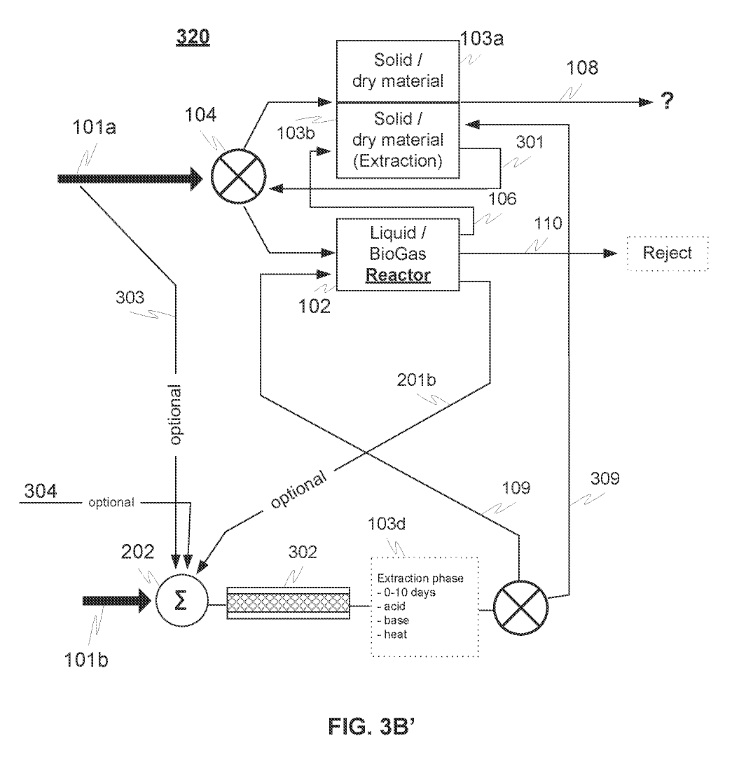

FIG. 2 illustrates another exemplary method and system 200 for producing biogas from waste stream 101, where the original waste stream feedstock is dry, like is the case with poultry waste for example. Again the system comprises at least one liquid waste container 102 (liquid reactor) for liquid fractions, and at least one solid waste container 103 (solid reactor) for solid fractions. The system also comprises a first separator 104 for at least partially separating liquid fraction from the waste stream 101 into the liquid waste container 102 (e.g. 1^{st} methane fermentation tank) and solid fraction from the waste stream 101 into the solid waste container 103 (e.g. 2^{nd} methane fermentation tank). After separation said solid waste container 103 advantageously comprises more solid content than the liquid waste container 103.

However, now the system 200 (for dry waste stream feedstock) comprises a mixer 202 before the first separator 104 in order to introduce the liquid from the liquid waste container 102 via a communications means 201 into the waste stream 101 and thereby to extract more liquid from the waste stream 101 in the separation 104. Thereby at least portion of the liquid fraction of the waste stream feedstock 101 and additionally also extracted liquid are separated at least partially by the separator 104, whereafter said liquids are transferred into the liquid waste container 102.

Advantages of the embodiment described in FIG. 2 are that the original feedstock is so dry that in the separations phase 104 the extraction of the liquid fraction would otherwise be very low, but by introducing some liquid into the waste stream in step 202, more liquid is achieved from the waste stream in step 104 for the liquid (fermentation) container 102 and thereby more powerful fermentation and biogas production is achieved.

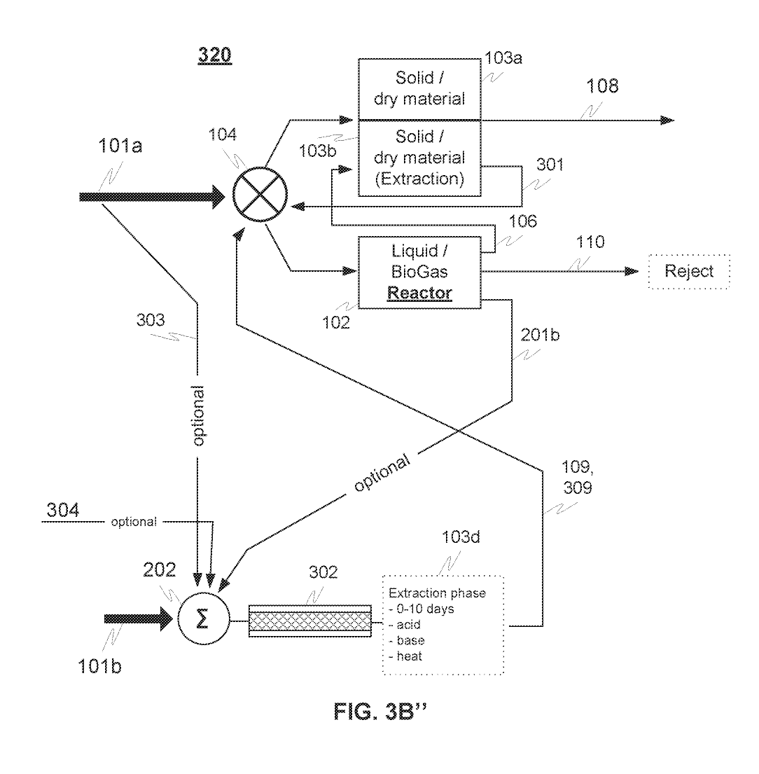

FIG. 3A illustrates another exemplary method and system 300 for producing biogas from waste stream 101. Now the system 300 is as a hybrid system comprising features from the both systems depicted in FIGS. 1-2, and therefore it can be applied for waste streams 101 with varying liquid vs. solid contents.

The system 300 (as well as also the other systems 100, 200) may additionally comprise a pH-sensor 301 for measuring pH-value of the liquid for example in the liquid waste container 102 and a controller 302 for controlling the introduction (recycling) of the liquid from the liquid waste container 102 to the waste stream portion comprising solid fraction via the mixers 105, 202. The controller 302 may be configured to use a pump (not illustrated) or other transferring means so that the introduction of the liquid is performed advantageously when the pH-value of the liquid in the liquid waste container 102 is essentially a certain predetermined value, whereupon the liquid is functioning as a buffer pH solution when introducing into the solid fraction stream or solid container.

In addition the systems 300 (or 100, 200) may also comprise a sensor 303 for measuring dry/wet content of the incident waste stream feedstock for example in connection with the first separator 104 or in connection with the mixer 202, 105, 112. Again the controller 302 may be configured to control the volume of the liquid to be introduced (recycled) from the liquid waste container 102 to said waste stream via the mixers 105, 112, 202 so that the liquid percentage of the waste stream after introduction is at an appropriate level, such as at least 60%, more advantageously at least 70%, or even more advantageously at least 80%.

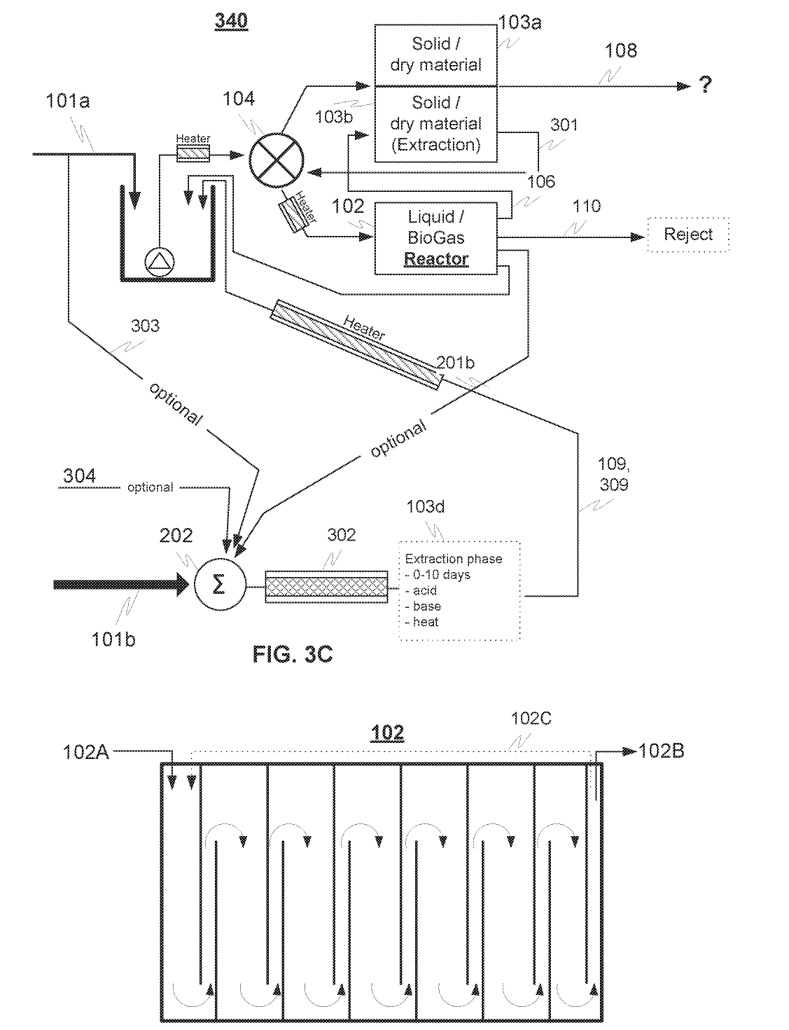

Furthermore the controller 302 may be configured to discharge at least portion of the liquid fraction from the liquid waste container 102 into a sequential liquid waste container (such as is depicted in connection with FIG. 4) or into a waste water treatment station. The controller 302 may be configured to discharge the liquid for example after 2-6 days, more advantageously after 2-4 days and even more advantageously after 2-3 days from the moment when the liquid fraction is separated into the liquid waste container 102, or after a certain pH threshold value is fulfilled or due to another triggering event.

In addition the systems 300 (or 100, 200) may also comprise a heating element 304 and heat controller 305 with suitable temperature sensor, which are configured to keep the temperature of the liquid in the liquid waste container in an appropriate temperature level in order to maximize the fermentation and biogas production.

Furthermore the systems 300 (or 100, 200) may also comprise anaerobic micro-organisms or microbes (e.g. Methanogenesis bacteria, like Saccharomyces cerevisiae) arranged advantageously in the liquid waste container 102 to interact with the liquid fraction and thereby producing biogas. According to an embodiment of the invention said anaerobic micro-organisms or microbes are arranged into a surface or surface arrangement having large surface area so that interaction of the microbes and the liquid and thereby the biogas production would be as effective as possible. The surface advantageously offers a solid support for the bacterial culture, and may be a sheet, a plastic pellet, sand, a biofilm, or the like promoting bacterial retention and increasing bacterial population. Other substances such as silica can also be added to the reactors to promote the chemical and biochemical reactions therein.

The container 102 may also comprise pump or propeller or the like configured to achieve liquid flow and thereby intensifying the interaction of the liquid with the microbes. According to an embodiment the surface(s) with said anaerobic micro-organisms may be arranged into plurality of units, such as floating units. Each units comprise advantageously maximal surface area for the micro-organisms, and according to an exemplary embodiment the surface may be implemented by a HUFO®-filter.

In addition FIGS. 3B′, 3B″ and 3C illustrates another embodiments 320, 340 of the invention, where the main parts and functions are similar than in other embodiments described in this document.

It is to be noted that the waste stream input 101 in the embodiments may comprise both a slurry waste stream input 101a and a dry manure waste input 101b. The dry manure waste may be e.g. poultry waste, and comprise in addition straw or grass material, peat, sawdust or the like, whereas slurry waste may be e.g. cattle waste, for example and not limiting only to those.

According to an embodiment the system may further comprise a ripper means 302 for ripping especially the dry manure waste 101b into smaller portions before introducing it in to the separator 104 or liquid waste container. Due to ripping the surface area of dry manure waste particles is increased, which enhances the fermentation process remarkably. As an example the ripper comprises gaps or other holes, blades or the like of diameters between 0.1-1.0 mm, advantageously elliptical gaps with minor axis between 0.1-0.3 mm, advantageously 0.2 mm, and with major axis between 0.5-0.8 mm, advantageously 0.6 mm.

The system may also comprise additional temporary solid waste container 103d after the ripper means 302, where the ripped solid waste material can be kept few days, such as 0-10 days, where the liquid or inoculum, as well as also acid, base or other substances enhancing and triggering the fermentation and extraction processes of the waste stream 101b can be added, before introducing said waste stream 101b to the separator 104 and the further process steps, for example. However, it is to be noted that the temporary solid waste container 103d is optional and that the waste stream 101b can be introduced to the separator 104 directly after ripper means 302.

Still according to an exemplary embodiment the system may further comprise the mixer 202 for introducing liquid 201b from the liquid waste container 102, slurry 303 from the slurry waste stream input 101a, and/or third liquid 304, such as water, and/or inoculum material into the dry manure waste stream 101b to introduce bacteria and other substances from the later phase of the previous process as a culture medium and thereby triggering and strengthen the fermentation process of the new incoming waste in its early stage or phase.

Again the system may comprise a heating means 305 also in connection with at least one communication means 106, 109, 201, 309 transferring liquid and/or solid portions in order to triggering or strengthen the fermentation and extraction process. According to an exemplary embodiment the liquid in the liquid container 102 is typically quite warm due to fermentation processes, for example, and the warn liquid may then be introduced into a temporary waste container 306 or other separator or mixer before said liquid container 102 (or upstream), whereupon the warm liquid may be used for warming or heating at least portion of said communication means transferring the waste streams. Again it is to be noted that according to an embodiment energy produced in the fermentation process or biogas process of the system can be used for heating in said heating means 305.

The temporary waste container 306 may be, as an example, a sludge tank with a suitable pump mechanism, and where e.g. liquid from the liquid containers and separators, as well as inoculum material from the later phases of the fermentation process can be introduced to the temporary waste container 306 to trigger said fermentation and biogas production process, as is illustrated elsewhere in this document.

According to an embodiment the liquid waste container 102 is arranged to function as a plug flow reactor, as is illustrated in FIG. 3D. The advantage of the plug flow reactor is that it can be used for concentrating the introduced liquid via anaerobic digestion without any essential blending of the liquid concentrations between the different phases and input 102A and output 102B ends of the plug flow reactor, whereupon the liquid with different concentrations from different phases can be taken and used for example as an inoculum material for different purposes needing different concentration. However, also other types of reactor can be used. Furthermore it is to be noted that there may be backflow 102C in the liquid waste container 102 (or reactor).

FIG. 4 illustrates an exemplary arrangement 400 according to the invention, where the arrangement comprises plurality of solid waste containers 103 as well as plurality of liquid waste containers 102, where each of the contents of the container may be in different fermentation state. For example the liquid may be kept first 2-4 days in the first liquid fermentation container 102a, next 2-4 days in the second first liquid fermentation container 102b, etc. The discharge may be implemented for example via a controller with a pump or it might also be gravity operated. Same applies also with the solid waste containers 103a, 103b, 103c.

In the FIG. 4 also a biogas collection pipe 401 and biogas containers 402 are illustrated, but naturally the other systems 100-300 also comprises means 401, 402 for collecting the produced biogas even though they are not depicted in FIGS. 1-3.

FIG. 5 illustrates an exemplary diagram 500 of methane fermentation rates according to an embodiment of the invention versus a prior art method, where it can be seen that the biogas production from the separated liquid fraction (in the liquid waste containers 102) according to the invention is very effective way to produce biogas very fast (curve 501). Especially it can be seen that during the first 2-4 days the production of the biogas from the liquid fraction is very fast, approximately 50% more than with the typical prior art biogas production from the original raw (non-separated) feedstock (curve 502). In addition it is to be noted that biogas is also produced in the solid waste container (curve 503), but the production rate is much lower than with the liquid fraction.

Thus when having at least 50% production within 2-4 days, the invention offers remarkable advantages over the known prior art, because the circulation period of the liquid in the containers can be kept within 2-4 days. Therefore there is no need for so big container volumes as with the prior art solution, where the raw feedstock must be kept in the containers at least 30-60 days. The typical prior art biogas reactor for 100 cattle requires about 30 m³ containers to keep the raw feedstock in the container for 30 days.

With the invention the requirements for the liquid waste container volume is only about 2-3 m³ (and for the solid waste container only about 1-2 m³) for the same amount of cattle, because the time requirements are only for 2-4 days.

According to the invention the systems 100-400 can be arranged into a moveable module, such as a shipping container or the like. The moveable main module may comprise for example the separators, mixers as well as biogas collection means and couple of liquid and solid waste containers. In addition, according to an embodiment, another moveable auxiliary module may comprise inlet means for connecting said auxiliary module to the outlet of the main module (or other auxiliary module), and comprising additional liquid and solid waste containers. Therefore for example a farm or ranch may be provided with one main moveable module and numbers of additional auxiliary containers depending on the requirements.

The invention has been explained above with reference to the aforementioned embodiments, and several advantages of the invention have been demonstrated. It is clear that the invention is not only restricted to these embodiments, but comprises all possible embodiments within the spirit and scope of the inventive thought and the following patent claims.

1-26. (canceled)

27. A system for producing biogas from waste stream comprising organic material,
wherein the system comprises:
a liquid waste container, and a solid waste container,
a separator for at least partially separating liquid fraction into the liquid waste container and solid fraction into the solid waste container from the waste stream so that said solid waste container comprises more solid content than the liquid waste container, and
a communication device between the liquid waste container and a mixer for introducing liquid from the liquid waste container to the waste stream portion comprising solid fraction.

28. A system according to claim 27, wherein the system comprises the mixer before the separator in order to introduce the liquid from the liquid waste container and/or inoculum material into the waste stream comprising biomass and to extract more liquid from said waste stream comprising biomass.

29. A system according to claim 27, wherein the system comprises the mixer between the separator and solid waste container in order to introduce the liquid from the liquid waste container into the solid waste container and to extract more liquid from the solid fraction contained in the solid waste container, whereupon the system also comprises:
a second separator in connection with the solid waste container for separating liquid fraction into the liquid waste container from the solid fraction of the solid waste container, and
a communication device between the second separator and at least one liquid waste container to introduce said liquid fraction separated from said solid fraction to said at least one liquid waste container.

30. A system according to claim 27, wherein the waste stream input comprises a slurry waste stream input and a dry manure waste input, whereupon the system further comprises a ripper for ripping the dry manure waste into smaller portions before introducing it in to the separator or liquid waste container.

31. A system according to claim 30, wherein the system comprises the mixer for introducing:
liquid from the liquid waste container,
slurry (303) from the slurry waste stream input, and/or
third liquid, such as water, and/or inoculum material, into the dry manure waste stream.

32. A system according to claim 27, wherein the system comprises heating device in connection with at least one communication device transferring liquid and/or solid portions.

33. A system according to claim 27, wherein the liquid waste container is arranged to function as a plug flow reactor for concentrating the introduced liquid via anaerobic digestion without any essential blending of the liquid concentrations between the different phases and input and output ends of the plug flow reactor.

34. A system according to claim 30, wherein the ripper comprises gaps of diameters between 0.1-1.0 mm, advantageously elliptical gaps with minor axis between 0.1-0.3 mm, advantageously 0.2 mm, and with major axis between 0.5-0.8 mm, advantageously 0.6 mm.

35. A system according to claim 27, wherein the system comprises pH-sensor for measuring pH-value of the liquid in the liquid waste container and a controller for controlling the introduction of the liquid from the liquid waste container to the waste stream portion comprising solid fraction so that the introduction is performed advantageously when the pH-value of the liquid from the liquid waste container is essentially a certain predetermined value.

36. A system according to claim 27, wherein the system comprises a sensor for measuring dry content of the incident waste stream, and a controller for controlling the volume of the liquid to be introduced from the liquid waste container to said waste stream via the mixer so that the liquid percentage of the waste stream after introduction is at least 60%, more advantageously at least 70%, and even more advantageously at least 80%.

37. A system according to claim 27, wherein the system comprises a mixer or introduction means for introducing acid, such as sulfuric acid, NaOH, Ca(OH)₂, enzyme or biocatalyst into the solid fraction.

38. A system according to claim 27, wherein the system comprises a surface comprising anaerobic micro-organisms/microbes in said liquid waste container configured to interact with said liquid fraction and thereby producing biogas, and/or wherein the surface with said anaerobic micro-organisms is arranged into plurality of units, each units comprising maximal surface area for said micro-organisms, and wherein said system is configured to introduce the liquid fraction to said surfaces of the units.

39. A system according to claim 27, wherein the system comprises a controller configured to discharge at least portion of said liquid fraction from said liquid waste container as an inoculum after 2-6 days, more advantageously after 2-4 days and even more advantageously after 2-3 days from the moment when said liquid fraction is separated into said liquid waste container.

40. A system according to claim 27, wherein the system comprises a mixer or introduction device for introducing additional organic waste material into the solid waste container, such as grass, reed, creed canary grass, or the like.

41. A system according to claim 27, wherein the system comprises plurality of the liquid waste containers in series, whereupon the system is configured to discharge the liquid from the previous liquid waste container into the subsequent liquid waste container after a certain time period and/or when the liquid in said previous liquid waste container comprises essentially a certain pH-value.

42. A system according to claim 27, wherein the system comprises a dry-type-reactor and a communication device between the solid waste container and said dry-type-reactor to introduce the solid material from the solid waste container into the dry-type-reactor, and wherein the system additionally comprises a mixer before the dry-type-reactor in order to introduce liquid from the liquid waste container into said dry-type-reactor.

43. A system according to claim 27, wherein the system comprises a heat treatment device for heating the content of liquid waste container and/or solid waste container.

44. A module for producing biogas from a waste stream comprising organic material, wherein the module comprises the system of claim 27.

45. A method for producing biogas from waste stream comprising organic material,
wherein the method comprises:
separating at least partially liquid fraction into a liquid waste container and solid fraction into a solid waste container from the waste stream so that said solid waste container comprises more solid content than the liquid waste container, and
introducing liquid from the liquid waste container to the waste stream portion comprising solid fraction in order to extract more liquid from said solid fraction.

46. A method according to claim 45, wherein the liquid from the liquid waste container is introduced into the waste stream comprising biomass before said separation in order to extract more liquid from said waste stream comprising biomass.

47. A method according to claim 45, wherein the liquid from the liquid waste container and/or inoculum material is introduced into the solid waste container in order to extract more liquid from the solid fraction contained in the solid waste container, the method further comprising separating liquid fraction into the liquid waste container from the solid fraction of the solid waste container.

48. A method according to claim 45, wherein the waste stream comprises a slurry waste stream and a dry manure waste, whereupon the dry manure waste is ripped by a ripper into smaller portions before introducing it into the separator or liquid waste container.

49. A method according to claim 48, wherein the method comprises steps of introducing:
liquid from the liquid waste container,
slurry from the slurry waste stream input, and/or
third liquid (304), such as water, and/or inoculum material, into the dry manure waste stream.

50. A method according to claim 45, wherein at least portion of the liquid fraction from the liquid waste container is discharged after 2-6 days, more advantageously after 2-4 days and even more advantageously after 2-3 days from the moment when said liquid fraction is separated into said liquid waste container.

51. A method according to claim 45, wherein acid, comprising sulfuric acid, NaOH, Ca(OH)₂, enzyme or biocatalyst, and/or additional organic waste material, comprising grass, reed, creed or canary grass, is introduced into the solid waste container.

52. A method according to claim 45, wherein plurality of the liquid waste containers are arranged in series, whereupon the liquid from the previous liquid waste container is discharged into the next liquid waste container after a certain time period and/or when the liquid in said previous liquid waste container comprises essentially a certain pH-value.
